(19) United States
(12) Patent Application Publication
(10) Pub. No.: US 2019/0311797 A1
(43) Pub. Date: Oct. 10, 2019

(54) DEVICE AND METHOD FOR MANAGING BOOKINGS OF APPOINTMENTS

(71) Applicant: SHOW SAVE, Bruxelles (BE)

(72) Inventor: Mohammed EL HAMROUNI, Bruxelles (BE)

(73) Assignee: SHOW SAVE, Bruxelles (BE)

(21) Appl. No.: 15/999,617

(22) PCT Filed: Feb. 16, 2017
(86) PCT No.: PCT/EP2017/000218

§ 371 Date: Aug. 20, 2018

(30) Foreign Application Priority Data
Feb. 18, 2016 (BE) BE/2016/0034

(51) Int. Cl. G16H 40/20; G06Q 10/02; G06Q 10/10

(52) U.S. Cl. CPC G16H 40/20; G06Q 10/1095; G06Q 10/02

(57) ABSTRACT

A device for managing bookings of appointments for a service, which device comprises a data processing unit having a memory for storing therein data comprising per person a parameter indicating a rate that the concerned person did not shown up at the appointments, which data processing unit comprises a generator provided for determining per person and on the basis of his parameter a probability that the person will not show up at the appointment, which data processing unit comprises a coupling member provided for coupling an action to said appointments on the basis of the parameter which has been allotted to the person, which coupling member comprises a memory provided for storing therein for each couple each time formed by one of the actions and one of the parameters a value indicating the reduction of the probability that the concerned person following the concerned action will not show up at a next appointment.

The present invention relates to a device for managing bookings of appointments for a service in a predetermined field, in particular for a medical visit, which device comprises a data processing unit connected to a first memory provided for storing therein data of persons having booked or would like to book an appointment, which data comprise per person a parameter indicating a rate that the concerned person did not shown up at the appointments in the predetermined field booked in the past, which data processing unit comprises a generator provided for determining per person and on the basis of his parameter a probability that the concerned person will not show up at the appointment the person wants to make, which generator is also provided for updating the parameter of the concerned person after that the time period foreseen for the booked appointment has lapsed and on the basis of an information indicating that the concerned person has shown up or not at the concerned appointment, which data processing unit comprises a coupling member having a second memory provided for storing therein a list of actions to be undertaken and related to the booking of appointments, which coupling member is provided for coupling one of said actions to said appointments on the basis of the parameter which has been allotted to the person.

The invention also relates to a method for managing bookings of appointments for services in each time a predetermined field.

Such a device and such a method are known from the patent application WO 2011/109496. According to the know device and method a parameter indicating a rate that the concerned person did in the past not show up at a booked appointment is allotted to each person of which the data are present in the first memory. This parameter is updated after that the moment at which the appointment of the concerned person has lapsed and in function of the fact that this person showed up or not at the appointment. Moreover, it is foreseen to allot to certain persons, of which the risk that they do not show up at the appointment is high, an action to undertake before the booked appointment should take place. This action will be allotted to the person of which the parameter indicates that this person has to be considered for such an action when booking the appointment. This action can for example be that the concerned person is imposed to telephone the day before the appointment for confirming his presence at the appointment, or to call this person.

The application WO 2014/071023 describes a device and a method for reducing the probability that a time range would remain unused due to the fact that the person does not show up at the booked appointment by offering the possibility of double booking.

A drawback of the known device and method is that they do not offer an effective means for evaluating the impact of all the actions mechanisms put in place.

The object of the invention is to realise a device and a method for managing bookings of appointments enabling to offer a means for evaluating the impact of the actions put in place.

To this purpose a device according to the invention is characterised in that the coupling member comprises a third memory provided for storing therein per couple each time formed by one of the actions and one of the parameters an indicative value of the reduction of the probability that the concerned person following the concerned action will not show up at a next appointment to be booked in the predetermined field, said processing unit being provided for updating that indicative value on the basis of the number of times that the concerned action has reduced the probability that the person did not show up at the next appointment. By allotting to each couple of an action and a parameter an indicative value of the reduction of the probability that the concerned person, following the concerned action, will not show up at a next appointment to be booked in the predetermined field, it becomes possible to evaluate the impact of each action. Thus, one obtains a tool enabling to verify the impact of the action and to update the indicative value of this action having proven that it contributes or not to reduce the probability that the person will not show up at the next appointment.

A preferred embodiment of the device according to the invention is characterised in that the third memory is structured as a matrix in order to store therein the actions and parameters according to rows and columns. This matrix structure enables to facilitate the access and the storage of the data in this third memory.

The invention will now be described in more details with respect to the drawings illustrating a preferred embodiment of the device and the method according to the invention. In the drawings:

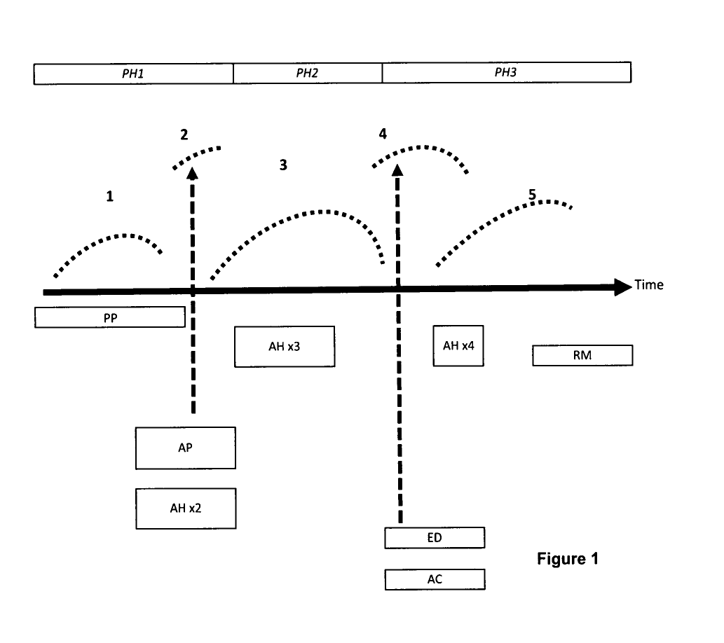

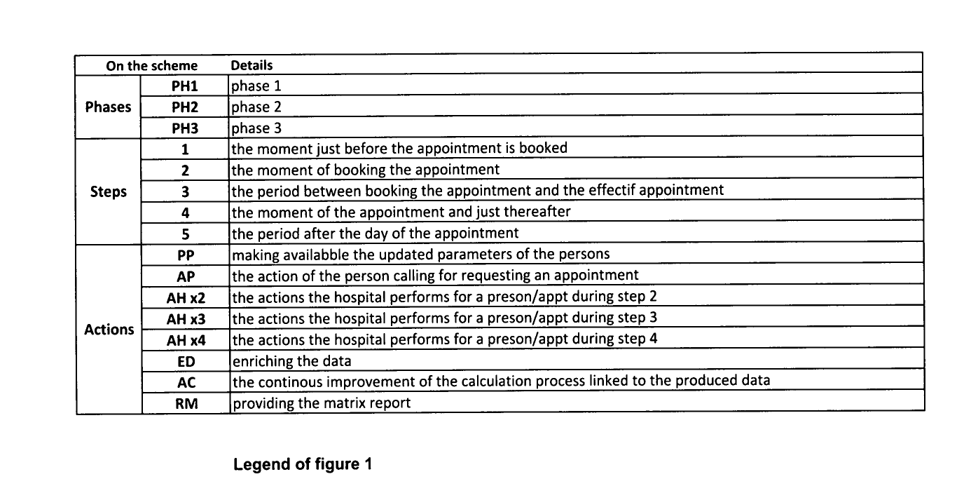

FIG. 1 illustrates the method of booking an appointment;

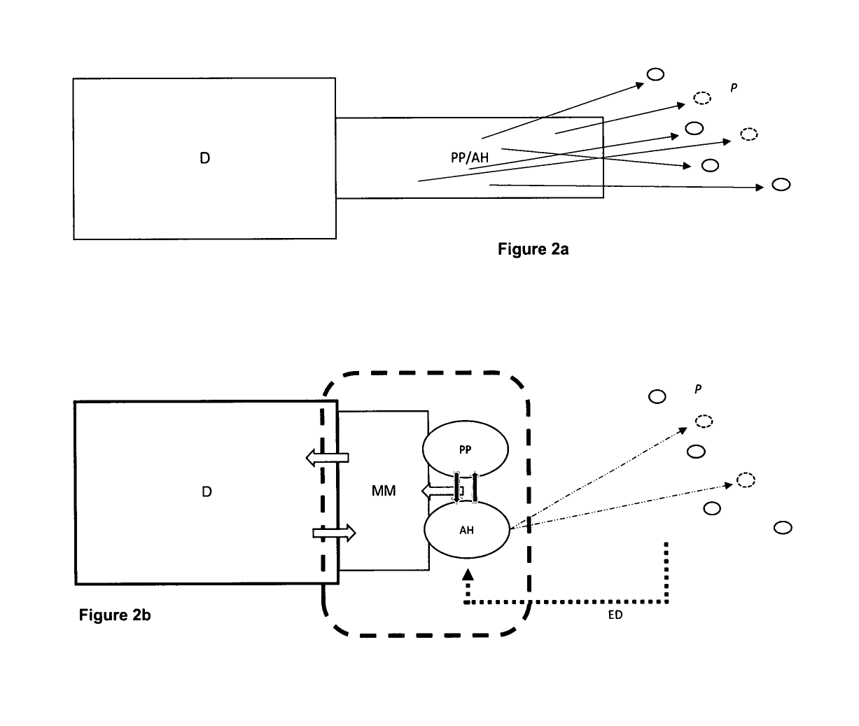

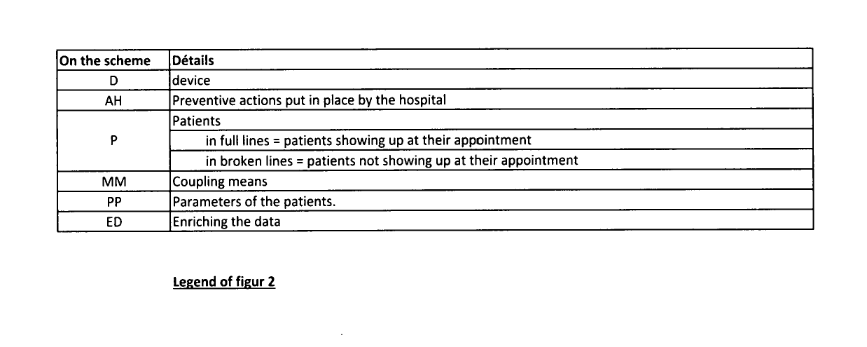

FIG. 2a illustrates schematically the prior art and FIG. 2b the invention;

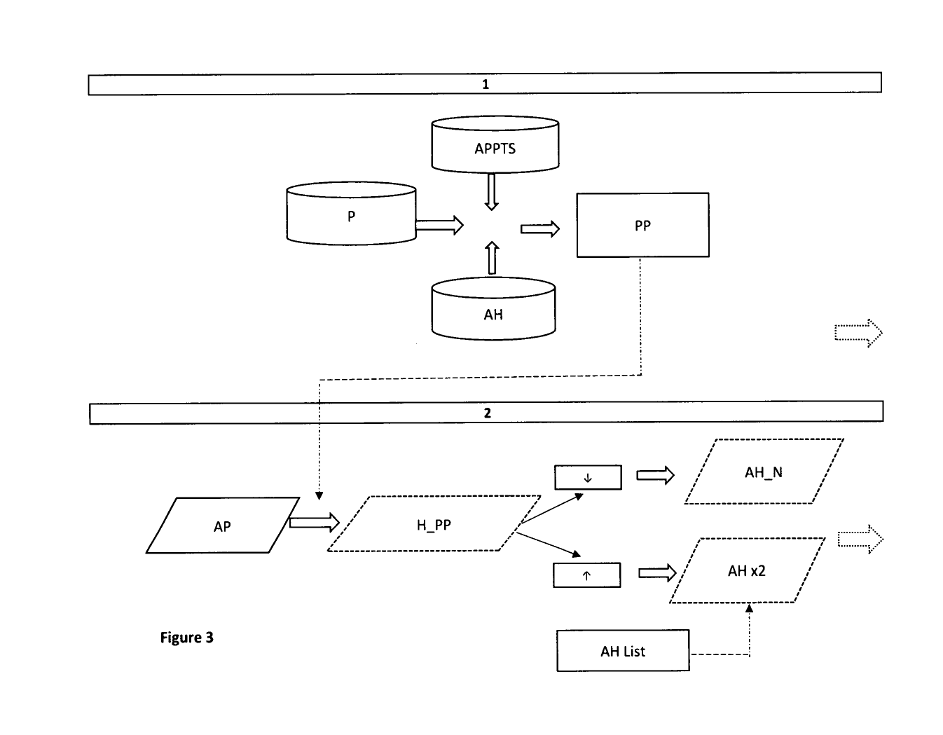

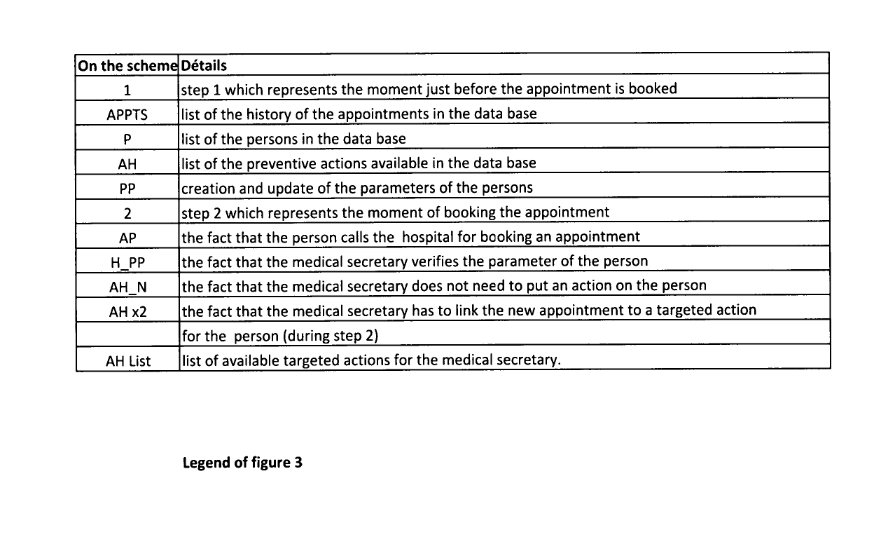

FIG. 3 illustrates the data processing by the data processing unit upon booking an appointment;

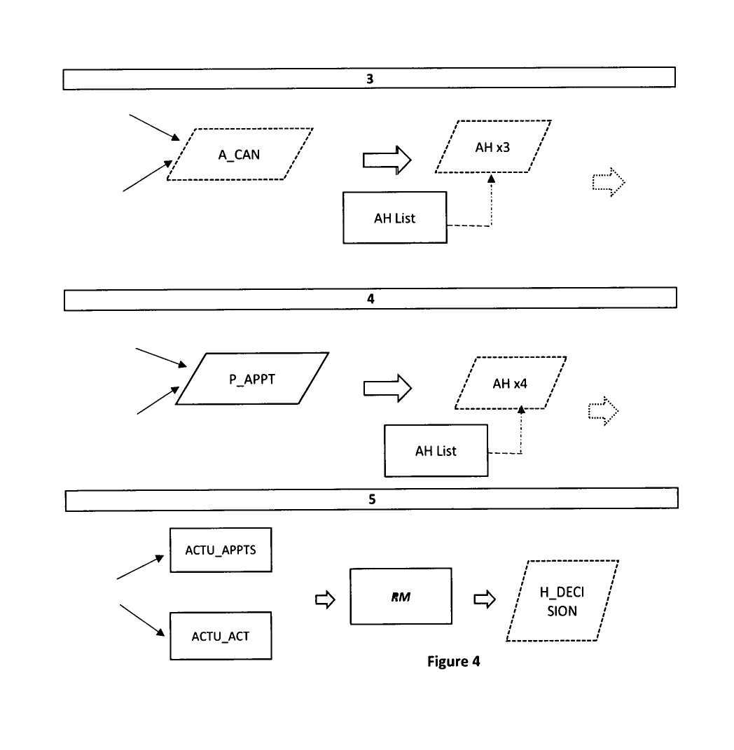

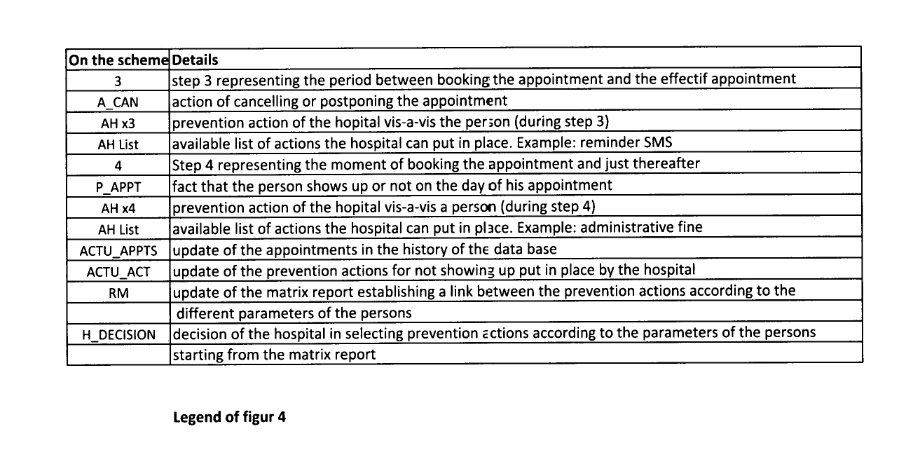

FIG. 4 illustrates the data processing by the data processing unit around the day of the appointment; and

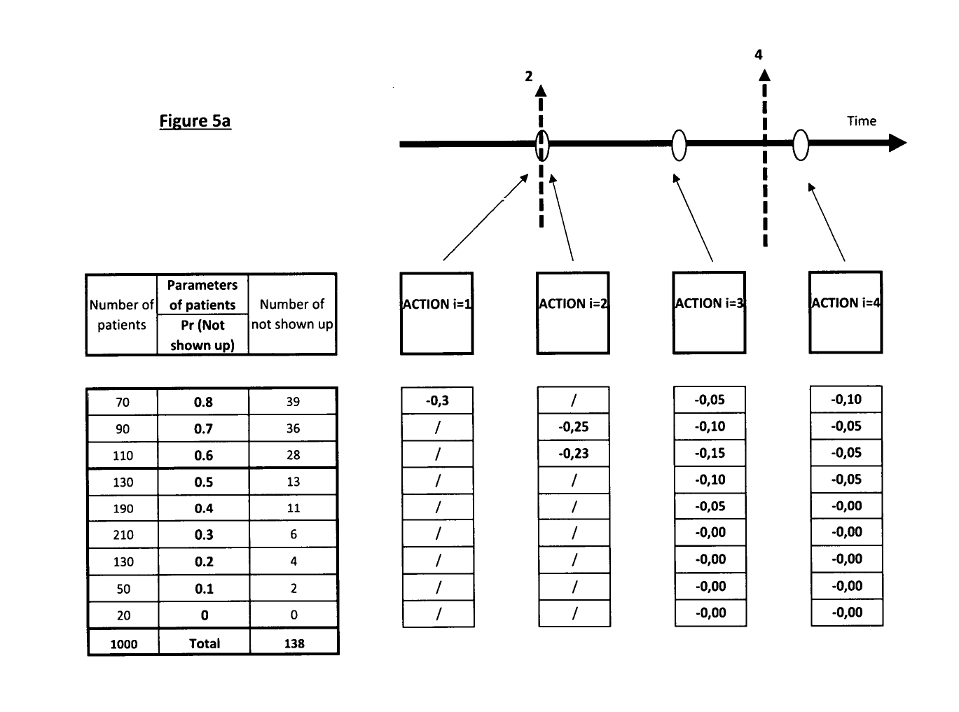

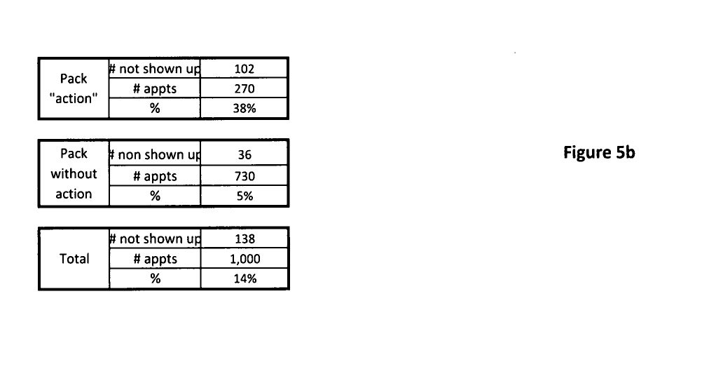

FIG. 5 illustrates the organisation of the third memory.

In the drawings a same reference has been allotted to a same or analogous element.

It is a well-known phenomenon that persons, who booked an appointment, do not show up at this appointment. This can be for example an appointment by a hair dresser, a bank, a physician or a dentist, or an appointment for an exam in a hospital. In particular in the medical field, the fact of not showing up at the booked appointment is frequent. This can have several reasons, i.e. that the person has forgotten it, that the person feels better and is of the opinion that a medical visit is no longer necessary, or simply the fear to go there. The probability of not showing up at the appointment is moreover not the same in different medical fields. So, dentists suffer from a higher probability than general practitioners, that the patient will not show up at the appointment. The probability varies also from person to person, so well organised persons will have less tendency to forget than less organised persons. Of course, the age of the person will also play a role.

Of course, this phenomenon is not a particularity of the medical field, but in this field, and in particular at the hospitals, the fact that the persons do not show up at the booked appointments causes severe logistical and economic consequences. So medical apparatuses cannot be used and the physicians or dentists lose a precious time waiting for a patient who will not show up.

For clarity reasons, the following description will be limited to the example of the medical field, in particular to hospitals, but it will be clear that the invention can be used in other fields where booking an appointment plays a role.

In order to remedy to this problem, the invention proposes a device and a method for managing the booking of appointments which not only has as an object to reduce the probability that a person does not show up at his booked appointment, but also to present to them information and means enabling to verify if the actions put in place for reducing the probability that a person does not show up at a next appointment carry their effects.

FIG. 1 illustrates the method for booking an appointment. In this appointment booking one distinguishes in general three phases;
The phase of booking the appointment, called phase 1 (PH1);
The phase in a period before the date of the appointment, called phase 2 (PH2); and
The phase of the appointment date, called phase 3 (PH3) and within a period after the appointment.

During the phase 1 of booking the appointment the person will contact (AP), for example by telephone, his physician, dentist or the hospital, in order to fix an appointment. It is known, that at the booking of this appointment, the person who will grant the appointment can already look in the data base of the device for consulting the parameters (PP) of the person who wishes to book an appointment. As illustrated in FIG. 2a, which illustrates the prior art, the device (D) put at the disposal of the physician, the dentist or the hospital, comprises a data base in which the parameters of the persons as well as a list of actions (AH) are stored. The person who will grant the appointment can consult this data base and, as the case may be, put in place an action vis-à-vis the person (P), who wishes to obtain this appointment. Alternatively, the person who will grant the appointment could allot (AHx2) an action to the hospital, the concerned physician or dentist. Those actions will be selected in function of the parameter of the concerned person. The way according to which those parameters and actions are determined will be described hereunder. The data base is regularly updated during that phase 1 in order to update i.a. the data of the patient. During phase 2, a few days before the appointment date, the person who received an action to undertake, so as for example calling for confirming the appointment, will have to execute this action for confirming his appointment, otherwise the appointment will be cancelled. When the hospital or the physician's or dentist's secretariat has to contact (AHx3) the person in order to remind him about his appointment, this action will be performed during phase 2. It has however to be noted that physicians or hospitals prefer that the persons call them, rather than that they have to call, as this latter action causes a high cost of which they prefer not to have to take it into charge. The day at which the appointment has to take place, phase 3 is started up. Either the person shows up at the appointment and his parameter will be adapted (ED) to allot him a positive credit, or the person does not show up and his parameter will be adapted (ED) to give him a negative credit. It is also possible that if the person did not show up at the appointment a fine is imposed (AHx4) to him. As the case me be the device can generate a report (RM) which will be furnished to the user of the device.

Measuring the impact of the allotted actions is not evident and increases considerably the work of the physician, dentist or hospital service. The invention proposes to this purpose a device and a method enabling to evaluate what has been the impact of the action on the behaviour of the person who has shown up or not at the appointment. FIG. 2b illustrates in a schematic way the device according to the invention. The device according to the invention comprises means (MM) which cooperate with the data base and the list of actions in order to enable an evaluation of the impact of the actions and thus to enrich (ED) the stored data.

In order to allot the parameter to each person who wishes to book an appointment, the device according to the invention comprises a data processing unit, which is illustrated in a schematic manner in FIG. 3. This data processing unit is for example formed by a microprocessor (not shown in the figure) which is connected to a memory P provided for storing therein the data of the persons who in the past have booked an appointment. The device comprises, or is connected to, a first data base APPTS where a historic of appointments booked in the past by the concerned person are stored. The device also comprises, or is connected to, a second memory AH where a list of actions is stored. To each of those actions a historic of the actions imposed in the past by the physician, the dentist or the hospital to the concerned person is allotted. This historic indicates how the persons have reacted to those actions. The historic is preferably stored in the second memory, but it is also possible to store it in the first memory. The device also comprises a generator PP for generating and updating the parameters of the persons on the basis of their behaviour that they show up or not to the appointment.

The data comprises per person a parameter indicating a rate that the concerned person did in the past not show up at a booked appointment. This parameter is for example calculated on the basis of the fact that on appointments booked in the past by the concerned person, that person showed up or not to the appointment. To this purpose the data stored in the first data base APPTS and in the first memory P are taken into account. The calculation of the parameter can be based either only on the showing up or not of the person at the appointment but can also take into account of other factors such as the age of the person, the type of appointment, the state of the patient, and so on. Thus, it is possible to make a distinction between an appointment with a dentist, a general practitioner, a treatment in a hospital, and so on. The fact that those factors are stored for each patient will then enable to determine the parameter in a more precise manner and to take them into account when the appointment is booked. A manner for determining this parameter is for example realised by using a Bayesian algorithm. The parameter allotted to each person is preferably stored in the first memory P with the other data of the patients.

It should be noted that the invention is not only applied to persons of which the parameter is already stored in the first memory P, but also to persons who book an appointment for the first time with this physician, this dentist or this hospital. In the latter case the data of that person are loaded in the first memory upon the first booking of the appointment and the parameter is also determined while booking this appointment. The determination of the parameter can for example be done by allotting a parameter of which the value is predetermined and is equal for each newcomer, or depends on certain criteria such as the age, the visited practice, and so on.

The data processing unit also comprises a generator, preferably integrated into the microprocessor, provided for determining per person and on the basis of his parameter a probability that the concerned person will not show up at the appointment he wishes to book. Thus, when during phase 1 the concerned person will book (AP) an appointment, the person who will grant him this appointment will consult (H_PP) the device by introducing therein the name or a reference of the person and the generator will then calculate this probability. If the probability is weak (↓) the appointment will be granted without anything more (AH_N). If, on the contrary, the probability is high (↑), this will be taken into account upon booking the appointment. This can for example be that the concerned person will be put in a time period at the end of the day or that a specific action will be imposed to him (AHx2). This action will be retrieved from the list (AH List) of actions stored in the second memory.

Hereunder a plurality of non-exhaustive examples of actions put in place for preventing that the patients will not show up at the booked appointment are illustrated:

| | | | |
|---|---|---|---|
| ACTIONS | Reminders | Reminders | Administrative |
| | SMS | by telephone | fines |
| Phase | Phase 2 | Phase 2 | Phase 3 |
| during | | | |
| which the | | | |
| action is | | | |
| done | | | |
| Action of | yes | yes | yes |
| the | | | |
| hospital | | | |
| EXAMPLES | On | For the | For the |
| OF | certain | new patients; | patients |
| Targeted | specialism | For the | having |
| STRATEGY | only; | appointments | missed: |
| | Without | detected as | their last |
| | targeting | having a risk | appointment; |
| | (SMS for | of not | their two |
| | all | showing up. | last |
| | patients). | | appointments. |

The hospitals using those prevention actions try to find an appropriate solution in terms of efficiency and cost. They try in such a manner to minimize on a long-term basis the fact that persons do not show up at the appointments. They also try to minimize the total costs linked to these preventive actions. Those costs are mainly linked to the constraint of re-contacting the persons (by SMS, by sending a mail for an administrative fine, via reminders by phone, and so on). In order to minimize those costs and as can be seen in the table hereabove, certain targeting techniques are used in certain cases.

As illustrated in FIG. 4 during phase 2 it can be that the person, the physician or the dentist has to cancel or shift (A_CAN) the appointment. If this is the case, the time of the appointment will be changed (AHx3) and if necessary the action will be adapted.

The generator is also provided for, during phase 3, update the parameter of the concerned person each time after that the time period foreseen for the scheduled appointment has lapsed (P_APPT). This update is realized on the basis of an information indicating that the concerned person has shown up or not at the concerned appointment. Thus, practically every day and in function of the fact that the persons have shown up at the booked appointment, the parameter of the concerned persons will be updated in order to keep the parameters stored in the memory up to date. If the no show up at the appointment has been established a fine can, as the case may be, be imposed to the patient (AHx4).

The data processing unit also comprises a coupling member (AH and H_DECISION) connected to the second memory where the list of actions to be taken and relating to the booking of an appointment is stored. The coupling member is provided for coupling, when the appointment is booked by a person and on the basis of his parameter, one of said actions to the appointment. Thus, for example if the parameter of the concerned person is high, the probability that the person will not show up at the appointment, for example for a dental control, will also be high and an action to confirm by telephone the appointment will be imposed to him.

The fact of coupling an action to the booking of an appointment will already as such enable to reduce the probability that the person does not show up at the appointment, as the object of this action is just to remind him about the appointment. In order to now enable to better evaluate the impact of such an action, the coupling member comprises a third memory provided for storing therein indicative values Vᵢⱼ of the reduction of the probability that the concerned person following the concerned action will not show up at the booked appointment. Those values are each time allotted to a couple formed by one of the actions I and one of the parameters j. To this purpose the third memory is preferably structured as a matrix in order to store therein the actions and the parameters by rows and columns, as illustrated in FIG. 5. One finds for example in in column i and row j, the value Vᵢⱼ allotted to the couple formed by the action stored in column i and the parameter stored in row j. In order to limit the size of the matrix the parameters are grouped by ranges of values, the ranges being mutually exclusive, the indicative values being allotted by range.

In the example shown in FIG. 5a one sees at the left a list with the values of the parameter j and in the upper part the actions is aligned. Thus, action i=1 is for example a request for confirming the appointment, action 1=2 a warning, action i=3 the sending of an SMS and action i=4 the imposing of a fine. The stored actions are thus as well those imposed on the person as those the hospital imposes them self. It will be clear that the number of actions is only given by way of an example and that a lower or higher number can also be allotted. The list with the values of the parameter is ordered in decreasing order, that means that the highest probability value that the person will not show up at the appointment is placed on top of the list. Preferably the list of parameters can also contain the number of patients to which this value has been allotted. Thus, one sees in the example of FIG. 5 that to seventy persons, on a total number of thousand persons, a value of 0.8 has been allotted as parameter, to ninety persons a value of 0.7 and so on. It is also possible to indicate in this list the number of times that the persons did not show up at the appointment. Thus, one sees in the example of FIG. 5 that for hundred and thirty-eight (138) appointments where the person did not show up, thirty-nine of those appointments related to persons having a parameter equal to 0.8.

Still according to the example of FIG. 5 one will see that the indicative value V₁₁=−0.3, which signifies that to the couple formed by the action i=1 and the parameter j=1 (0.8) the indicative value −0.3 has been allotted. This signifies that if the action to confirm the appointment is imposed on a person having a parameter equal to 0.8, his parameter will be reduced by 0.3 and thus the probability that he will show up at a later appointment will be increased. One also sees that the indicative value V₆₃=−0.00, which signifies that to the couple formed by action i=3 and the parameter j=6 (0.3) the indicative value −0.00 has been allotted. This signifies that if there is imposed on the hospital for a person having a parameter equal to 0.3 the action of sending him an SMS will not reduce his parameter and thus that this action has so to say no effect. Starting from those indicative values, the hospital is able to optimize the actions in the sense of targeting them according to the different allotted parameters. So, in the example hereabove, one can see the differentiating efficiency of the prevention actions. The values Vᵢⱼ are preferably determined by self-learning algorithms which calculate them for each action in function of the fact that the person did show up or not at the appointment and of his parameter.

For persons with a very high risk (parameter=0.8) that they will not show up at the appointment, the hospital has an interest to link to those persons an action i=1 (request for confirming the appointment) which is the most efficient action for increasing the probability that they will show up at their next appointment. Thus, when booking an appointment, the medical secretary imposes the person to confirm again his appointment “x” days before the appointment day, otherwise the appointment will be automatically cancelled. For persons with a high risk (parameter=0.7) that they will not show up at the appointment, the hospital has an interest to link to those persons an action i=2 (warning) which is the most efficient action for increasing the probability that they will show up at their next appointment. Thus, when booking an appointment, the medical secretary will warn the person that, if he does not show up at his next appointment, a more constraining measure for future appointments will be taken with respect to him. For persons with a small risk (parameter ≤0.4) that they will not show up at the appointment, the hospital has an interest to apply no targeted actions on those persons, as the probability that they will show up at the appointment is high enough.

Moreover, through this matrix, the hospital can directly visualize the impact of reminders by SMS. Those are useless for the persons with a small risk that they will not show up at the appointment and they are not efficient enough for persons with a high risk that they will not show up at the appointment.

One can consider that the hospital management will replace the non-targeted action consisting of sending a reminder by SMS by the hospital and which necessitates an activity of the hospital towards the person, and thus a cost for the hospital, by better targeted actions according to the risk profile of the persons.

Starting from this matrix of parameters and actions, the hospital management is in better shape to create and select targeted actions shaped according to their performance result on the different parameters allotted to the patients and which can be seen within the matrix. Moreover, it is possible to integrate within this matrix supplementary information elements which are of interest for the decision of the hospital management. For example, the image obtained by the realized targeted action, the costs of those targeted actions (financial and organizational), the long-term evolution of the parameters of the persons, and so on.

The set of actions put in place will also produce a global financial impact on the company where it will used. One can estimate the latter by calculating the differentiate financial impacts of the targeted actions (or non-targeted) according to the created patient profiles. Indeed, each action enables to realize savings in terms of increasing effective working time periods of which certain had large probabilities of being under-used without putting in place these actions.

Those savings can also be measured via different methodologies. For example, one can estimate, according to the profile of the patients, the additional number of appointments realized thanks to having imposed an action “x” with respect to the number of appointments realized if not accompanied by this action “x”. The obtained difference between those two values represents the number of additional appointments obtained by putting in place actions. Different sampling methods can also be used.

The number of additional appointments estimated hereabove is multiplied by the economical value of each of the appointments (potential business volume, costs of deployed resources, . . . ) for obtaining the economic impact obtained by the targeted actions for each patient profile. This will then also provide an estimation of the financial impact of an action/profile of the patients. Those financial impacts can be refined by adding also other factors such as for example the whole of the costs linked by putting in place the actions.

The processing unit is provided for updating this indicative value on the basis of the number of times that the concerned action has reduced the probability that the person did not show up at the appointment. Thus, if the data processing unit establishes that action i did not reduce the probability that the person shows up at the appointment, a lower indicative value will be attributed thereto thereby taking care that this action will be less used or even no longer used.

To this purpose and as illustrated in FIG. 4 under step 5 the device will, after the moment of the appointment has passed (ACTU_APPTS) actualize the first memory for indicating if the person has shown up or not at the appointment. The device will also actualize (ACTU_ACT) the actions for indicating the effect that they had on the persons and this by taking into account their parameter. The device will then actualize (RM) the matrix for actualizing the link between the action and the parameter. This actualization will then enable to adapt the selection (H_DECISION) of the actions.

FIG. 5b illustrates a report furnished by the device. This report shows that for the one hundred thirty-eight persons who did not show up at the appointment, it is useful to distinguish in a predictive manner the persons for whom a preventive action is necessary (102 persons of the 270 have a parameter situated between 0.6 and 0.8) from those for whom a preventive action is not necessary (36 persons of the 370 have a parameter situated between 0.5 and 0). Thus, those who decide in the hospital can, by means of that report (RM), use the targeted actions and optimized over time, of which they will observe the efficiency of the actions on the persons having a parameter with a higher value than the others. They can thus concentrate their efforts of targeting only on those persons of which the risk that they do not show up at the appointment is high (102/270 persons) and thus avoid to impose actions which are “useless” on the others (36/730 persons).

The device and the method according to the invention integrate the risk that the patient will not show up at the appointment as a targeting strategy enabling to visualize the differentiate impacts of all the prevention actions put in place according to the set of parameter values allotted to the persons. The advantage of this method or this device is that it enables to compare all the actions put in place according to an efficiency criterion which is the long-term behavior of the persons vis-à-vis the fact that they do not show up at the booked appointment. The update of the parameters of the set of persons and the availability of this information in the hospital system (phase 1) is coupled to a set of predetermined actions, leaving the freedom to the hospital for creating new actions in whatever phase (1,2 or 3), and differentiate according to different values of the parameters of the persons.

It also enables to estimate and update the parameter over all the variables (show up/not show up of patients at their past appointments, history of the patients, specialty used in the past, age, sex, domicile, . . . ) but also those linked to actions which have been put in place (or not) vis-à-vis that patient in the past (types of actions, date at which the action was put in place, . . . ). This targeting strategy also enables the hospital to visualize the efficiency of a more important number of targeted actions as they are realized at the time when the appointment is booked (phase1). Those actions are also coupled to certain parameters of the persons. That moment at which the targeted actions are put in place does not require that the hospital gets in touch with the person and minimize thus the cost of all the targeted actions.

1. A device for managing bookings of appointments for a service in a predetermined field, in particular for a medical visit, which device comprises a data processing unit connected to a first memory provided for storing therein data of persons having booked or would like to book an appointment, which data comprise per person a parameter indicating a rate that the concerned person did not shown up at the appointments in the predetermined field booked in the past, which data processing unit comprises a generator provided for determining per person and on the basis of his parameter a probability that the concerned person will not show up at the appointment the person wants to make, which generator is also provided for updating the parameter of the concerned person after that the time period foreseen for the booked appointment has lapsed and on the basis of an information indicating that the concerned person has shown up or not at the concerned appointment, which data processing unit comprises a coupling member having a second memory provided for storing therein a list of actions to be undertaken and related to the booking of appointments, which coupling member is provided for coupling one of said actions to said appointments on the basis of the parameter which has been allotted to the person, characterised in that the coupling member comprises a third memory provided for storing therein per couple each time formed by one of the actions and one of the parameters an indicative value of the reduction of the probability that the concerned person following the concerned action will not show up at a next appointment to be booked in the predetermined field, said processing unit being provided for updating that indicative value on the basis of the number of times that the concerned action has reduced the probability that the person did not show up at the booked appointment.

2. The device for managing as claimed in claim 1, characterised in that the third memory is structured as a matrix in order to store therein the actions and parameters according to rows and columns.

3. The device for managing as claimed in claim 1, characterised in that the coupling member is provided for forming the couples by value ranges of the parameters, the ranges being mutually exclusive, the indicative values being allotted per range.

4. The device for managing according to claim 1, characterised in that the indicative value indicates the reduction rate of the parameter to which it is coupled.

5. A method for managing bookings of appointments for services in each time a predetermined field, in particular appointments for medical visits, which method comprises the storage in a first memory of data of persons having booked or would like to book an appointment, which data comprise for each person a parameter indicating a rate that the concerned person has not shown up at the appointments in the predetermined field booked in the past, which method comprises the determination per person and on the basis of his parameter of a probability that the concerned person will not show up at the appointment the person wants to make, which method also comprises the update of the parameter of the concerned person each time after that the time period foreseen for the booked appointment has lapsed and on the basis of a received information indicating that the concerned person has shown up or not at the concerned appointment, which method comprises the storage in a second memory of a list of actions to be taken and relative to the booking of appointments, which method comprises, when the appointment is booked by one of said persons, the selection on the basis of the parameter of the concerned person of one of said actions and the allotment of the selected action to the booked appointment, characterised in that it comprises the forming of couples formed by each time one of the actions and one of the parameters and the allotment to each couple of a value indicative of the reduction of the probability that the concerned person following the concerned action would not show up at a next appointment to be booked, said indicative value being updated on the basis of the number of times that the concerned action has reduced the probability that the person did not show up at the booked appointment.
